(19) United States
(12) Patent Application Publication
(10) Pub. No.: US 2024/0374873 A1
(43) Pub. Date: Nov. 14, 2024

(54) Automated Guidewire Extraction Systems and Methods

(71) Applicant: Bard Access Systems, Inc., Salt Lake City, UT (US)

(72) Inventors: Joe Spataro, Cottonwood Heights, UT (US); Kyle G. Thornley, Farmington, UT (US)

(21) Appl. No.: 18/661,387

(22) Filed: May 10, 2024

Related U.S. Application Data
(60) Provisional application No. 63/465,942, filed on May 12, 2023.

(51) Int. Cl. A61M 25/09; A61M 25/06

(52) U.S. Cl. CPC A61M 25/09041; A61M 25/0625; A61M 25/0631; A61M 25/065; A61M 2025/09125

(57) ABSTRACT

Catheter placement assemblies having automatic guidewire disengagement can include a handle and seal housing assembly defining a needle lumen and guidewire lumen. Once the vasculature is accessed, the guidewire advances through the handle guidewire lumen to a target location. The needle is withdrawn proximally through the handle needle lumen with a first force. When the needle tip is withdrawn into the seal housing, the seal housing locks to the needle tip. Applying a second, greater force urges the seal housing to disengage the handle. The seal housing is retained in place by a detent and/or timing lock mechanism. As the seal housing is urged proximally, a support pin retains the guidewire in place causing the guidewire to automatically disengage the seal housing. The seal housing mitigates needle stick injuries and allows the needle and seal housing assembly to be removed, allowing for unobstructed advancement of the catheter over the guidewire.

### PRIORITY

This application claims the benefit of priority to U.S. Provisional Patent Application No. 63/465,942, filed May 12, 2023, which is incorporated by reference in its entirety into this application.

### BACKGROUND

Central venous catheter (“CVCs”) are commonly introduced into patients and advanced through their vasculatures for placement by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are rapidly insertable central catheter (“RICC”) insertion assemblies, introducer-needle assemblies, and methods that address the foregoing. Notably such RICC insertion assemblies, introducer-needle assemblies, and methods include needle-stick-injury protection, as set forth below.

### SUMMARY

Disclosed herein is a catheter placement system including, a catheter, a needle supported by a needle hub and extending distally to a needle tip, the needle defining lumen, a guidewire, a seal housing including a tip shield transitionable between a locked position and an unlocked position, a tether extending between the needle hub and the seal housing, a handle defining a recess and configured to slidably receive the seal housing therein, wherein the handle and the seal housing co-operate to define a handle needle lumen and a handle guidewire lumen, and a detent configured to retain the seal housing within the recess when a first force is applied to the needle, and configured to deflect radially outwards to allow the seal housing to disengage the handle when a second force is applied.

In some embodiments, the first force is less than a threshold force required to deflect the detent, and the second force is equal to or greater than the threshold force.

In some embodiments, one or both of the first force and the second force urges the needle in a proximal direction.

In some embodiments, the tip shield in the locked position prevents distal movement of the tip of the needle relative to the seal housing, the tip shield being biased towards the locked position.

In some embodiments, the seal housing further includes a slot extending between an outer surface of the seal housing and one or both of the handle needle lumen and the handle guidewire lumen and configured to allow the portion of the guidewire to pass therethrough as the seal housing disengages the handle.

In some embodiments, the handle needle lumen extends between a distal end and a proximal end of the handle and seal housing assembly and is configured to slidably receive a portion of the needle therein, and the handle guidewire lumen communicates with the handle needle lumen and extends at an angle therefrom.

In some embodiments, the seal housing includes a seal formed of a compliant material and defining a fluid tight seal extending about a junction between the handle needle lumen and the handle guidewire lumen.

In some embodiments, the needle includes a guidewire aperture communicating with the needle lumen and configured to align with the handle guidewire lumen when the needle hub engages a proximal end of the handle.

In some embodiments, the needle further includes a slot extending between the guidewire aperture and the needle tip.

In some embodiments, the catheter is a RICC and further includes a blood flash system coupled to the needle hub.

In some embodiments, the catheter placement system further includes a support pin disposed within the handle and abutting against a portion of the guidewire to support the guidewire. The support pin causes the guidewire to automatically disengage the seal housing when the seal housing is urged proximally.

In some embodiments, the tether is configured to allow the needle hub to be withdrawn proximally up until a predetermined distance (d) where the tether prevents further proximal movement relative to the seal housing beyond the predetermined distance (d).

Also disclosed is a method of placing a catheter including, forming an insertion site using a needle supported by a needle hub, advancing a guidewire through a handle guidewire lumen of a handle, to a target location, withdrawing the needle proximally through a handle needle lumen until a distal tip is located within a seal housing, the seal housing defining a junction between the handle needle lumen and the handle guidewire lumen, transitioning a tip shield to a locked position to prevent further distal movement of the needle tip relative to the seal housing, and urging the needle proximally to deflect a detent radially outwards and disengage the seal housing from the handle.

In some embodiments, withdrawing the needle proximally further includes applying a first proximal force, and urging the needle proximally to deflect the detent includes a second proximal force, the second proximal force being greater than the first proximal force and equal to or greater than a threshold force required to deflect the detent.

In some embodiments, the seal housing is slidably engaged within a recess defined by the handle, the detent abutting against a portion of the seal housing to retain the seal housing within the recess when the first proximal force is applied.

In some embodiments, the step of urging the needle proximally urges a portion of the guidewire, disposed within the handle guidewire lumen, through a slot extending between the handle guidewire lumen and an outer surface of the seal housing.

In some embodiments, the method further includes a tether extending between the needle hub and the seal housing and configured to prevent further proximal movement of the needle hub relative to the seal housing beyond a predetermined distance (d).

Also disclosed is a catheter placement system including, a needle defining a needle lumen extending between a needle hub and a needle tip, a guidewire, a seal housing, a tether extending between the needle hub and the seal housing, a handle defining a recess and configured to slidably receive the seal housing therein, wherein the handle and the seal housing co-operate to define a handle needle lumen and a handle guidewire lumen, a tip shield transitionable between an unlocked position and a locked position, and a timing lock disposed within the seal housing and transitionable between a first position and a second position, wherein, in the first position, the timing lock is configured prevent movement of the seal housing relative to the handle, and wherein, in the second position, the timing lock is configured to allow the seal housing to slide relative to the handle.

In some embodiments, the timing lock is biased towards the second position and is retained in the first position by a portion of the needle disposed in a distal portion of the handle needle lumen.

In some embodiments, the timing lock is configured to transition from the first position to the second position when the needle tip is disposed within the seal housing.

In some embodiments, the timing lock in the second position allows the needle and seal housing assembly to disengage from the guidewire and handle assembly.

In some embodiments, the catheter placement system further includes a support pin coupled with the handle and configured to support the guidewire as the seal housing slides proximally relative to the handle. The support pin causes the guidewire to automatically disengage the seal housing when the seal housing is urged proximally.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings.

FIG. 1A shows a perspective view of a catheter placement system in an unfolded configuration, in accordance with embodiments disclosed herein.

FIG. 1B shows a plan view of a catheter placement system in a folded configuration ready for use, in accordance with embodiments disclosed herein.

FIG. 2 shows a side view of a catheter of the catheter placement system of FIG. 1A in an unfolded configuration, in accordance with embodiments disclosed herein.

FIG. 3A shows close-up detail of a distal portion of the catheter of FIG. 2, in accordance with embodiments disclosed herein.

FIGS. 3B and 3C show cross-section views of the catheter of FIG. 3A, in accordance with embodiments disclosed herein.

FIG. 4A shows a perspective view of a handle, needle and guidewire assembly, in accordance with embodiments disclosed herein.

FIG. 4B shows a longitudinal cross-section view of a handle, needle and guidewire assembly, in accordance with embodiments disclosed herein.

FIG. 5A shows a perspective view of a needle and seal housing assembly disengaged from a guidewire and handle assembly, in accordance with embodiments disclosed herein.

FIG. 5B shows a longitudinal cross-section view of a needle and seal housing assembly disengaged from a guidewire and handle assembly, in accordance with embodiments disclosed herein.

FIGS. 6A-6D show an exemplary method of use for a catheter placement system including a handle having a tip shield mechanism, in accordance with embodiments disclosed herein.

FIGS. 7A-7C show an exemplary method of use for a catheter placement system including a timing lock mechanism, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or “proximal section” of, for example, a catheter includes a portion or section of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal section, or the proximal length of the catheter can include the proximal end of the catheter; however, the proximal portion, the proximal section, or the proximal length of the catheter need not include the proximal end of the catheter. That is, unless context suggests otherwise, the proximal portion, the proximal section, or the proximal length of the catheter is not a terminal portion or terminal length of the catheter.

With respect to “distal,” a “distal portion” or a “distal section” of, for example, a catheter includes a portion or section of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal section, or the distal length of the catheter can include the distal end of the catheter; however, the distal portion, the distal section, or the distal length of the catheter need not include the distal end of the catheter. That is, unless context suggests otherwise, the distal portion, the distal section, or the distal length of the catheter is not a terminal portion or terminal length of the catheter.

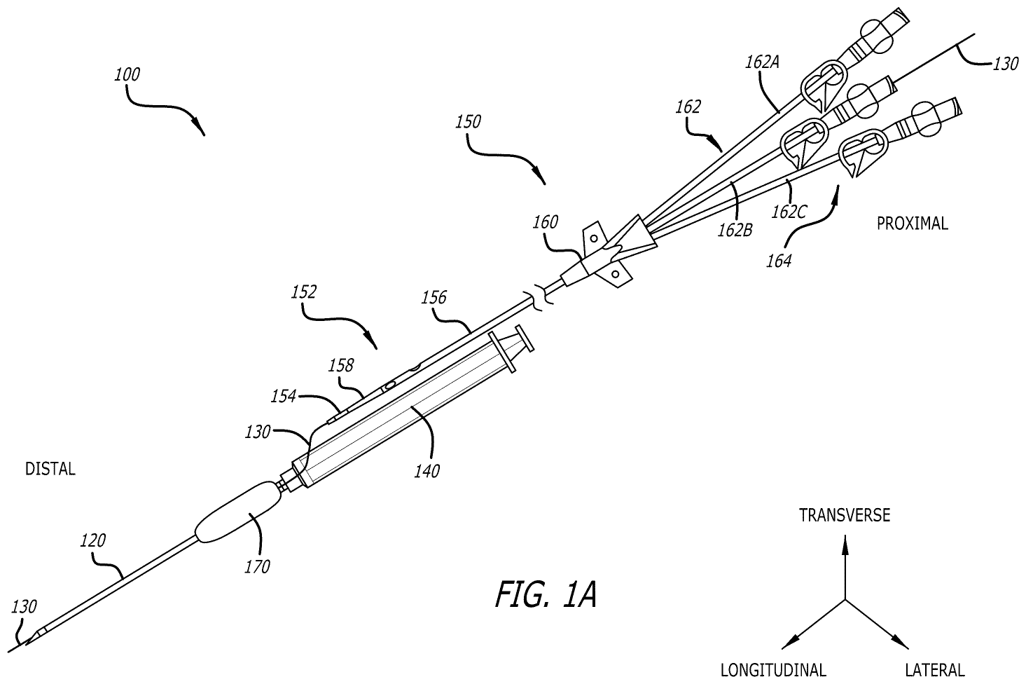

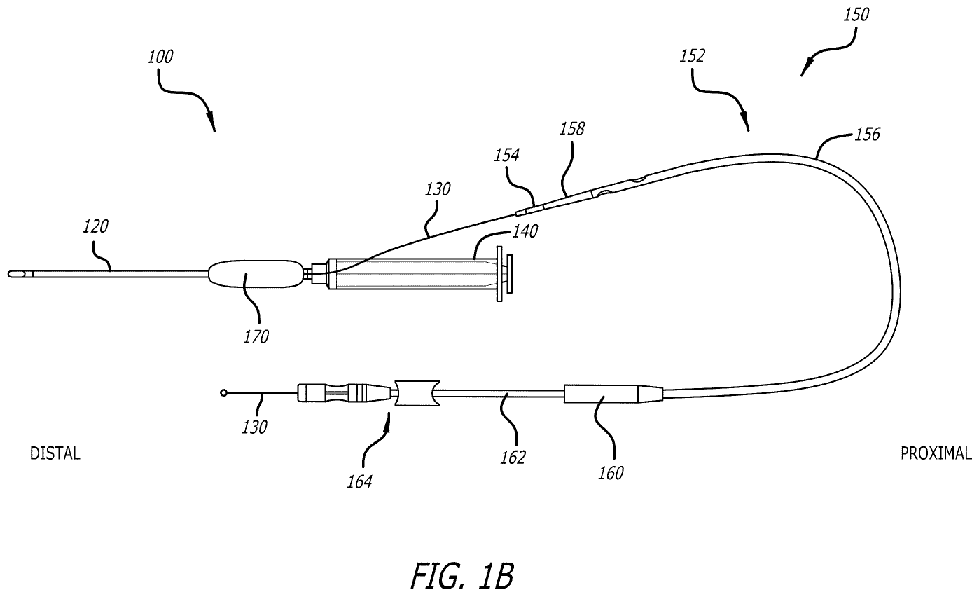

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes. A horizontal plane can be defined by the lateral axis and the longitudinal axis. A vertical plane extends normal to the horizontal plane.

Unless defined otherwise, all technical and scientific terms used herein have the same meaning as commonly understood by those of ordinary skill in the art.

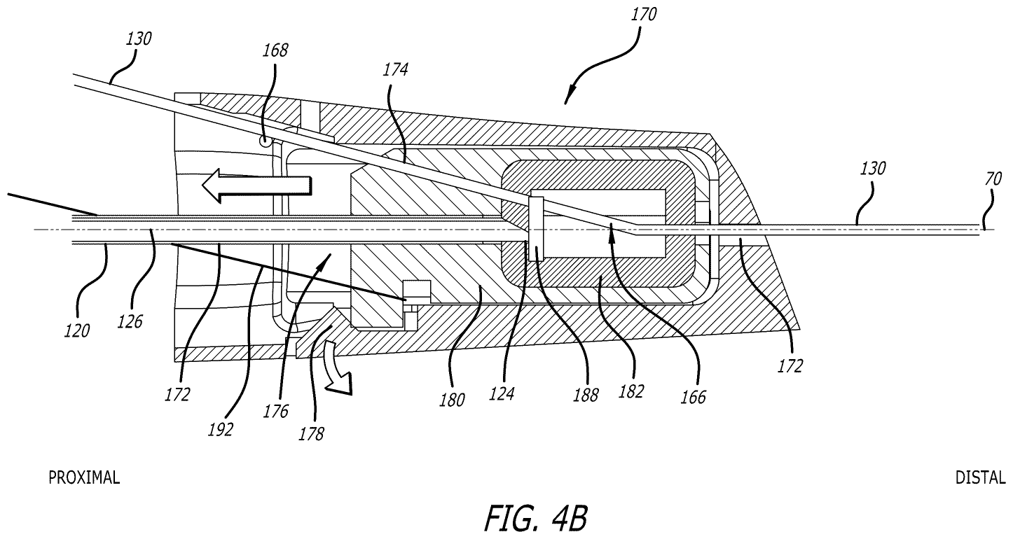

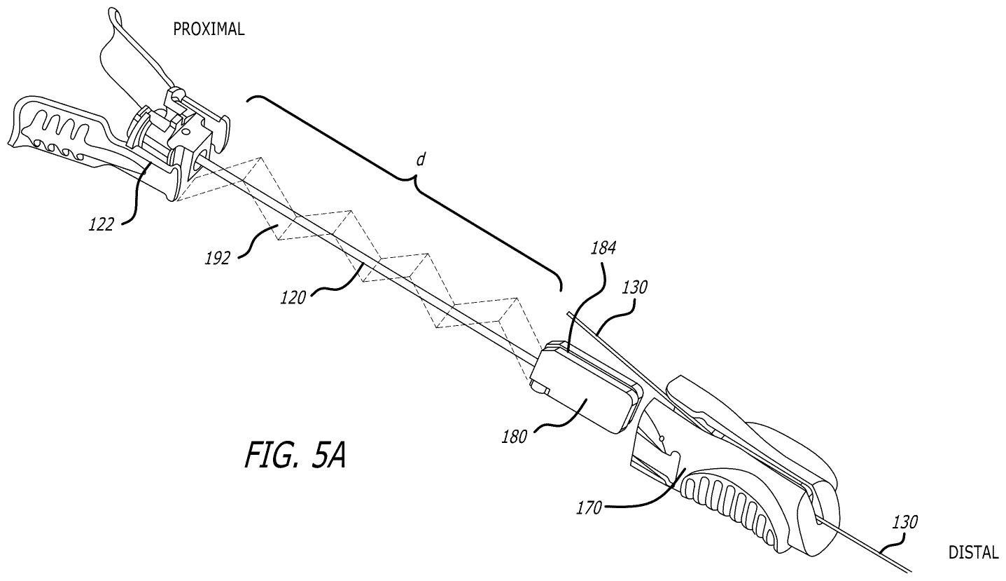

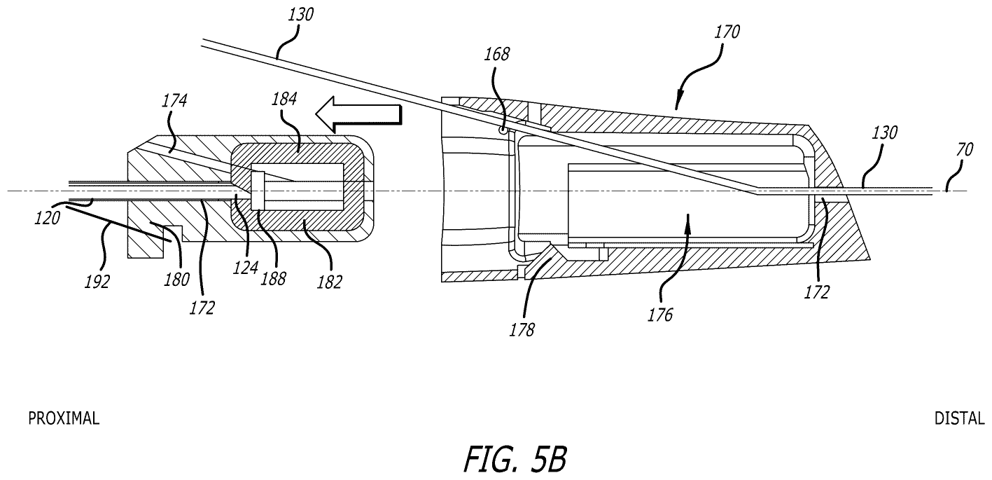

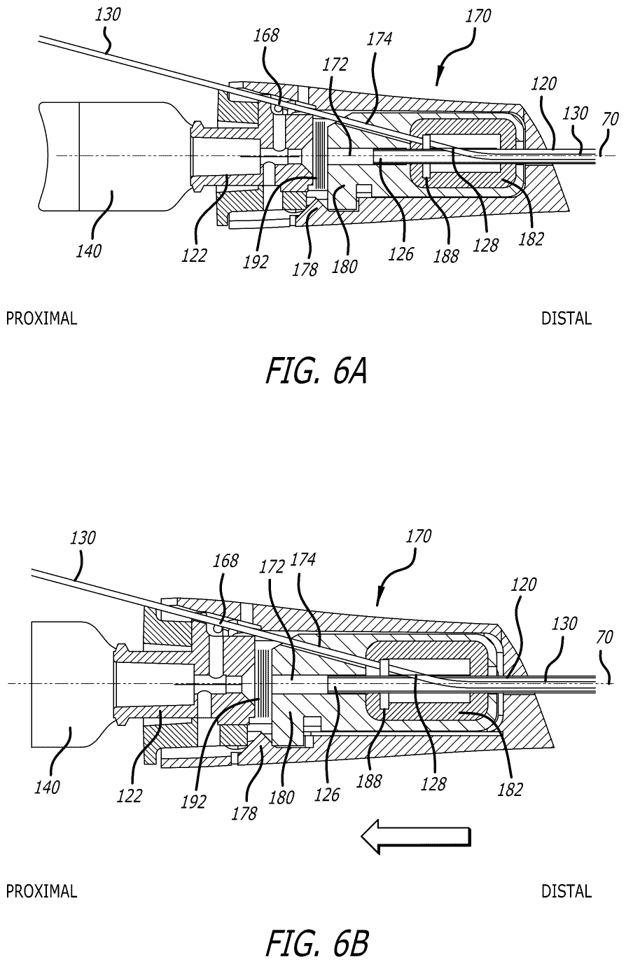

FIGS. 1A-1B show a catheter placement system (“system”) 100, generally including a needle 120, a guidewire 130, a blood flash system 140, a catheter 150, and a handle 170, which handle 170 is simplified for case of illustration. (See FIGS. 4A and 5A for additional details of the handle 170.) FIG. 1A shows the system 100 in an unfolded configuration, and FIG. 1B shows a plan view of the system 100 in a folded configuration ready for use. In an embodiment the catheter placement system 100 is a Rapidly Insertable Central Catheter (RICC) placement system 100 configured to place a RICC 150. However, it will be appreciated that other catheter placement systems configured to place other types of catheters are also contemplated. Exemplary catheters 150 include peripheral intravenous (PIV) catheters, peripherally inserted central catheter (PICC), central venous catheters (CVC), midline catheters, dialysis catheters, single lumen catheters, multi-lumen catheters, or the like.

In an embodiment, the catheter 150 generally includes a catheter body 152 supported at a proximal end by a catheter hub (“hub”) 160. The hub 160 includes one or more extension legs 162 extending proximally therefrom and includes a fluid coupling device, such as a luer lock 164, disposed at a proximal end of one of the one or more extension legs 162. The Luer lock 164 is configured to couple the extension leg 162 with a medical fluid line, syringe, or the like. Each extension leg of the one or more extension legs 162 is in fluid communication with a lumen of the catheter body 152. For example, a first extension leg 162A is in fluid communication with a proximal lumen 114A, a second extension leg 162B is in fluid communication with a medial lumen 114B, and a third extension leg 162C is in fluid communication with a distal lumen 114C. The catheter body 152 includes an access section 154 disposed distally, a catheter section 156 disposed proximally, and a dilation section 158 disposed therebetween. The access section 154 defines a single lumen and has a first outer diameter, the catheter section 156 defines two or more lumen and has a second diameter larger than the first diameter. The dilation section 158 disposed between the access section 154 and the catheter section 156 defines a tapered outer profile extending from the first diameter of the access section 154 to the second diameter of the catheter section 156. A guidewire 130 extends through a lumen of the catheter 150 from a proximal end of an extension leg 162, to a distal tip of the access section 154.

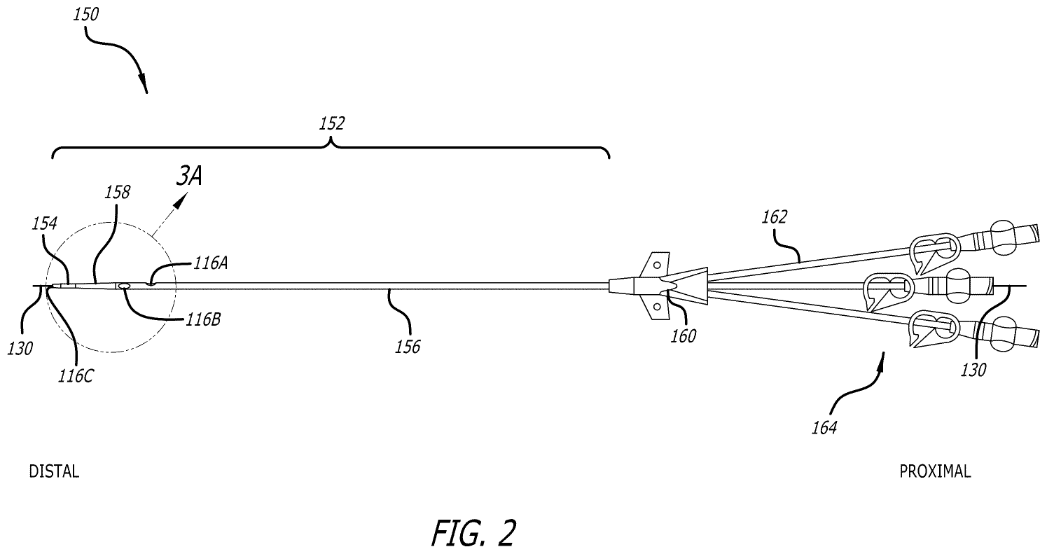

FIG. 2 shows further details of an exemplary catheter 150 of the system 100. In an embodiment, different sections of the catheter 150 are required to perform different functions and as such are required to display different mechanical properties. For example, the access section 154 and the dilation section 158 provide a more rigid mechanical properties or include a harder durometer material relative to the catheter section 156. As such, the access section 154 and dilation section 158 can withstand greater axial forces without kinking or collapsing, as these sections are urged distally, forming and dilating the insertion site. The catheter section 156 is formed of a softer durometer, or a more compliant material to facilitate negotiating the catheter section 156 through tortuous vascular pathways.

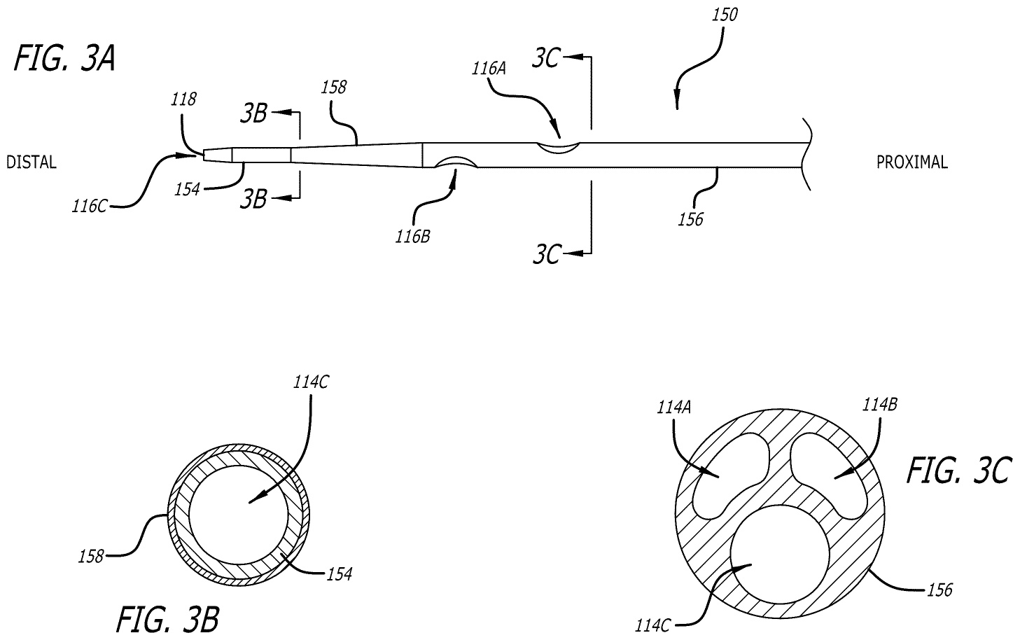

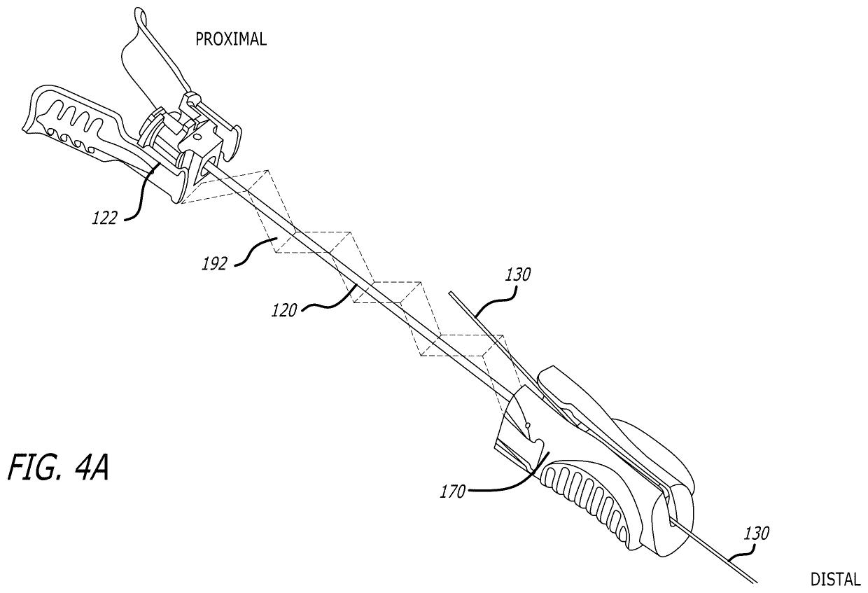

FIGS. 3A-3C show further details of a distal portion of the catheter 150, including the access section 154, the catheter section 156, and the dilation section 158. In an embodiment, the catheter section 156 includes a proximal lumen 114A terminating at a proximal lumen aperture 116A, and a medial lumen 114B terminating at a medial lumen aperture 116B. In an embodiment, each of the proximal lumen aperture 116A and the medial lumen aperture 116B extends through a side wall of the catheter section 156. In an embodiment, each of the proximal lumen aperture 116A and the medial lumen aperture 116B are disposed proximally of the dilation section 158. In an embodiment, the proximal lumen aperture 116A are disposed proximally of the medial lumen aperture 116B. In an embodiment, the proximal lumen aperture 116A and the medial lumen aperture 116B are disposed equidistant from a distal tip of the catheter 150.

FIG. 3B shows a cross section view of the catheter body 152 at point “A” of FIG. 3A. As shown, the access section 154 defines a single lumen and a relatively smaller outer diameter. In an embodiment, a proximal portion of the access section 154 is received within a distal portion of the dilation section 158. A distal lumen 114C of the catheter 150 extends to a distal tip 118 of the catheter 150 and communicates with a distal lumen aperture 116C. FIG. 3C shows a cross section view of the catheter section 156 at point “B” of FIG. 3A, showing the proximal lumen 114A, medial lumen 114B and distal lumen 114C.

Details on the catheter body, distal tip of the catheter, and methods of forming the catheter body and distal tip of the catheter can be found in U.S. Pat. No. 11,890,429, in U.S. Publication Nos.: US 2022/0001138, US 2023/0126869, US 2023/0132903, US 2023/0233796, US 2023/0233800, and US 2024/0091501, and in U.S. patent application Ser. No. 18/076,169, each of which is incorporated by reference in its entirety into this application. Further detail regarding catheter materials can be found, for example, in PCT/US2024/011269, filed Jan. 11, 2024, which is incorporated by reference in its entirety into this application.

Further details of catheter placement systems 100 in accordance with the embodiments described herein can be found, for example, in U.S. Pat. Nos. 11,517,719, 10,376,675, U.S. 2019/0255294, U.S. 2021/0069471, U.S. 2021/0113809, U.S. 2021/0113810, U.S. 2021/0121667, U.S. 2021/0121661, U.S. 2021/0228843, U.S. 2021/0283381, U.S. 2021/0322729, U.S. 2021/0361915, U.S. 2021/0330941, U.S. 2021/0330942, U.S. 2021/0402149, U.S. 2021/0402142, U.S. 2022/0032013, U.S. 2021/0402153, U.S. 2021/0379336, U.S. 2021/0283368, U.S. 2022/0062528, U.S. 2022/0032014, U.S. 2022/0126064, U.S. 2022/0193378, U.S. 2022/0176081, U.S. 2022/0193376, U.S. 2022/0193377, U.S. 2022/0152368, U.S. 2022/0176082, U.S. 2022/0193379, U.S. 2022/0296862, U.S. 2022/0323723, U.S. 2022/0370762, U.S. 2022/0362524, U.S. 2023/0043989, U.S. 2023/0041261, U.S. 2023/0039733, U.S. 2023/0042898, each of which is incorporated by reference in its entirety into this application.

FIGS. 4A-4B show further details of the handle 170 of the system 100. FIG. 4A shows a perspective view of the handle 170 including the guidewire 130 extending therethrough, and the needle 120 supported by the needle hub 122 in a retracted position. FIG. 4B shows a longitudinal cross-section view of the handle 170 of FIG. 4A. In an embodiment, the needle 120 defines a needle lumen 126 and further includes a needle guidewire aperture 128 extending through a wall of the needle 120 and communicating with the needle lumen 126.

In an embodiment, the handle 170 defines a recess 176 extending distally from a proximal end of the handle 170 and configured to receive a seal housing 180 therein. With the seal housing 180 located within the recess 176, the seal housing 180 co-operates with the handle 170 to define a handle needle lumen 172 and a handle guidewire lumen 174. The seal housing 180 is slidably engaged with the recess 176 such that it can be removed proximally from the recess 176 as described in more detail herein.

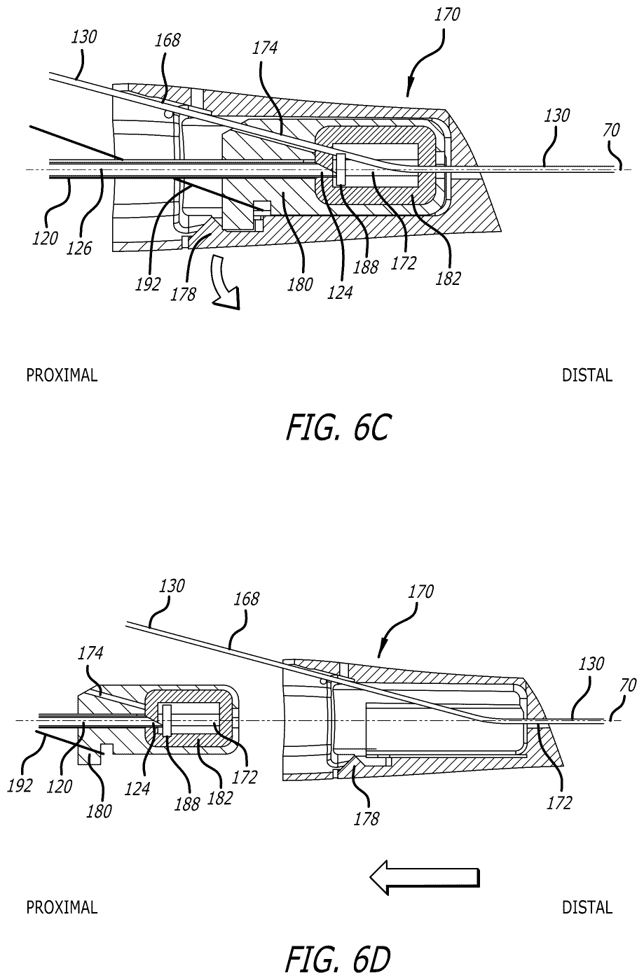

In an embodiment, the handle 170 and seal housing 180 co-operate to define a handle needle lumen 172 extending between a distal end and a proximal end of the handle 170 and configured to receive a portion of the needle 120 therethrough. The needle 120 is slidably engaged with the handle needle lumen 172 between a distal position (e.g., FIGS. 6A-6B) and a proximal position (e.g., FIG. 4B). The handle 170 and seal housing 180 assembly further define a handle guidewire lumen 174 communicating with the handle needle lumen 172 at a junction 166 and extending proximally at an angle therefrom. In an embodiment, with the needle 120 disposed in a distal position, the needle guidewire aperture 128 aligns with the junction 166. The guidewire 130 extends through the handle guidewire lumen 174, through the needle guidewire aperture 128 and into the needle lumen 126 and extends from a distal end of the needle tip 124 and/or distal end of the handle needle lumen 172.

In an embodiment, the handle 170 further includes a support pin 168 extending laterally and disposed below a portion of the guidewire 130. The support pin 168 is positioned above a top-most surface of the seal housing 180. As such, the support pin 168 does not obstruct the path of the seal housing 180 as the seal housing 180 is retracted from the recess 176. The support pin 168 is configured to support a portion of the guidewire 130 as the seal housing 180 is urged proximally.

In an embodiment, the seal housing 180 further includes a seal 182 formed of a compliant material and forming a seal extending about the junction 166 where the handle guidewire lumen 174 communicates with the handle needle lumen 172. The seal 182 is formed from a polymer, elastomer, rubber, silicone rubber, self-sealing silicone, or the like. As such, as one or both of the needle 120 and the guidewire 130 is withdrawn from the seal 182, the seal 182 self-seals providing a fluid tight seal and preventing any fluids disposed therein from escaping.

In an embodiment, the seal housing 180 further includes a tip shield mechanism (“tip shield”) 188 configured to transition between a first, or unlocked, position and a second, or locked, position. In the unlocked position, the tip shield 188 allows the needle 120 to slide relative to the seal housing 180, through the handle needle lumen 172. In the locked position, the tip shield 188 extends between the needle tip 124 and the distal end of the seal housing 180 to prevent any distal movement of the needle 120 relative to the seal housing 180. In an embodiment, the tip shield 188 is biased towards the locked position and is held in the unlocked position by the presence of a portion of the needle 120 in a distal end of the handle needle lumen 172. As the needle tip 124 is withdrawn into the seal housing 180 and thereby removed from a distal portion of the handle needle lumen 172, the tip shield 188 is allowed to transition to the locked position, preventing the needle tip 124 from extending distally from the seal housing 180.

In an embodiment, the system 100 further includes a tether 192 extending between the needle hub 122 and the seal housing 180. The tether 192 can be coupled to one or both of the needle hub 122 and the seal housing 180 using an adhesive, welding, ultrasonic welding, bonding, mechanical attachment (e.g., eyelet engaging a protrusion or hook), combinations thereof, or the like. The tether 192 can be formed from an organic or synthetic flexible material with a relatively high tensile strength. Exemplary materials can include metals, alloys, plastics, polymers, composites, wires, ropes, strings, tapes, combinations thereof, or the like. As shown, the tether 192 includes a pleated material that includes one or more apertures, allowing the needle 120 to extend therethrough. However, this is not intended to be limiting.

The tether 192 can transition between a folded position (FIG. 6A) and an unfolded position (FIGS. 4A, 5A). The tether 192 is configured to allow proximal movement of the needle hub 122 relative to the seal housing 180 up until a predetermined distance (d). Once the needle hub 122 has reached the predetermined distance (d) from the seal housing 180, the tether is fully unfolded and is in a stretched taught position (FIG. 5A) and prevents any further proximal movement of the needle tip 124, and the needle 120, relative to the seal housing 180. To note, the tensile strength of the of the tether 192 is configured to prevent any further movement of the needle hub 122 beyond the predetermined distance (d). Further, with the needle hub 122 at distance (d) the needle tip 124 is located within the seal housing 180 at a position that allows the tip shield 188 to transition to the locked position, preventing any distal movement of the needle 120 relative to the seal housing 180. As such, the needle tip 124 is locked within the seal housing 180, with the tip shield 188 preventing any distal movement and the tether 192 preventing any proximal movement. The needle 120, seal housing, tether 192 and needle hub 122 assembly can then be detached from the handle 170, as described in more detail herein.

In an embodiment, the handle 170 includes a detent 178 extending into the recess 176 and configured to retain the seal housing 180 within the recess 176. The detent 178 is supported by a flexible portion of the handle 170. As such, when the seal housing 180 is urged proximally using a first force, the detent 178 prevents the proximal movement of the seal housing 180 up until a second force, which is equal to or greater than a threshold force, is applied. When the proximal force exceeds the threshold force, the detent 178 deflects radially outwards with respect to a central longitudinal axis 70. With the detent 178 deflected outwards, the seal housing 180 can slide proximally and disengage the handle 170.

FIGS. 5A-5B show further details of the seal housing 180 and needle 120 assembly disengaged from the handle 170 and guidewire 130 assembly. FIG. 5A shows a perspective view and FIG. 5B shows a longitudinal cross-section view. In an embodiment, a first force is applied to the needle 120 in a proximal direction to withdraw the needle 120 through the handle needle lumen 172, and the tether 192 deploys transitioning from the folded configuration to the unfolded configuration. As the needle hub 122 approaches the predetermined distance (d) from the seal housing 180, the needle tip 124 is withdrawn into the seal housing 180. Just before the needle hub 122 reaches the predetermined distance (d) the needle tip 124 is withdrawn proximally of the tip shield 188 allowing the tip shield 188 to transition to the locked position. The needle tip 124 is then locked within the seal housing 180, and optionally within the seal 182 with further distal movement prevented by the tip shield 188. With the needle hub 122 at the predetermined distance (d) and the tether 192 fully unfolded, the user can apply a second force in a proximal direction to the needle 120 and seal housing 180 assembly. The second force is greater than the threshold force and can deflect the detent 178 radially outwards allowing the seal housing 180 to slide distally out of the recess 176, disengaging the handle 170. The seal housing 180 locks around the needle tip 124 and as such, the seal housing 180 prevents needle stick injuries from occurring once the needle 120 is disengaged from the handle 170. For additional detail regarding how the seal housing 180 locks around the needle tip 124, see the needle-stick-injury (NSI) protection mechanism set forth in PCT/US2024/011269, filed Jan. 11, 2024, which is incorporated by reference in its entirety into this application.

In an embodiment, the seal housing 180 includes a slot 184 extending longitudinally along a vertical plane and communicating between an outer surface of the seal housing 180 and the handle guidewire lumen 174. When the second force is applied and the seal housing 180 is urged proximally relative to the handle 170, a portion of the guidewire 130 that is disposed within the handle guidewire lumen 174 automatically disengages the handle guidewire lumen 174 and passes through the slot 184 to disengage the seal housing 180. To note, the support pin 168 supports the portion of the guidewire 130, as the portion of the guidewire 130 is urged through the slot 184, and mitigates any movement or kinking of the guidewire 130. As such, as the seal housing 180 disengages the handle 170, the guidewire 130 is automatically disengaged from the seal housing 180 but otherwise remains in place in the handle 170 and, optionally, a vessel of a patient if the distal end of the guidewire is disposed therein.

FIGS. 6A-6D show an exemplary method of use for the system 100. As shown in FIG. 6A, the needle 120 is in a distal position with the needle hub 122 engaged with a proximal end of the handle 170. The tether 192 is in the folded position disposed between the needle hub 122 and the seal housing 180. A portion of the guidewire 130 extends through the handle guidewire lumen 174, through the seal 182 and into the handle needle lumen 172. To note, the needle 120 includes a guidewire aperture 128 that aligns with the handle guidewire lumen 174 when the needle 120 is in a distal position (i.e., when the needle hub 122 is engaged with a proximal end of the handle 170). As such the guidewire 130 extends from the handle guidewire lumen 174 through the guidewire aperture 128 of the needle 120 and into the needle lumen 126, which in turn is disposed within a distal portion of the handle needle lumen 172.

Initially, the user advances the needle tip 124 to form an insertion site and access a vessel. The guidewire 130 is then advanced through the needle lumen 126 into the vessel to anchor the insertion site. As shown in FIG. 6B, a first proximal force is applied to the needle hub 122, urging the needle 120 proximally relative to the seal housing 180. Due to the friction between the needle 120 and the seal housing 180 and associated structures (e.g., seal 182, inner surface of the handle needle lumen 172, etc.) the seal housing 180 may be urged proximally until the seal housing 180 engages the detent 178. The first force is less than the threshold force required to deflect the detent 178, as such, the detent retains the seal housing 180 within the recess 176 of the handle 170 while allowing the needle 120 and needle hub 122 to continue to slide proximally. In an embodiment, the needle 120 includes a slot extending from the guidewire aperture 128 of the needle 120 to a distal tip 124 thereof to allow the guidewire 130 to disengage the needle 120 as the needle 120 is withdrawn proximally. In an embodiment, the needle 120 further includes a tearable sheath disposed on an outer surface thereof and configured to mitigate any fluids leaking from the needle lumen 126 through the slot. Further details and embodiments of the slotted needle can be found in U.S. application Ser. No. 17/970,005 filed Oct. 20, 2022, and U.S. application Ser. No. 17/971,182 filed Oct. 21, 2022, each of which are incorporated by reference in their entirety into this application.

As shown in FIG. 6C, the needle 120 is withdrawn proximally relative to the seal housing 180 and the tether 192 transitions from the folded configuration to the unfolded configuration, until the needle hub 122 is at the predetermined distance (d) relative to the seal housing 180. At which point, the needle tip 124 is disposed within the seal housing 180 and further movement of the needle 120 relative to the seal housing 180 is prevented, as described herein. For example, by a tip shield 188 and a tether 192 extending between the seal housing 180 and the needle hub 122. With the needle tip 124 positioned as shown in FIG. 6C, the tether 192 is stretched taught preventing any further proximal movement between the needle 120 and the seal housing 180. In an embodiment, when the needle tip 124 is withdrawn into the seal housing 180 a tip shield 188 is released. The tip shield 188 is biased towards the locked position and held in the open position by a portion of the needle disposed within a distal end of the handle needle lumen 172. As the needle tip 124 is withdrawn into the seal housing, the tip shield 188 is released and transitions to the locked position. In the locked position, the tip shield 188 engages the needle 120 and locks the seal housing to the needle tip 124.

With the needle tip 124 within the seal housing 180, a second force is applied to the needle 120, urging the needle 120, and seal housing 180, proximally. The second force is greater than the first force and is greater than a threshold force that is required to deflect the detent 178 radially outwards. As such, as shown in FIG. 6D, the seal housing 180 with the needle tip 124 disposed therein slides proximally and disengages the handle 170. As the seal housing 180 is urged proximally to disengage the handle 170, the portion of the guidewire 130 disposed within the handle guidewire lumen 174 is urged through the slot 184 to disengage the seal housing 180 without withdrawing the guidewire 130 from the vasculature.

Advantageously, the support pin 168 stabilizes the guidewire 130 as the portion of the guidewire 130 is urged through the slot 184, thereby mitigating kinking or movement of the guidewire 130. As such, the guidewire 130 is automatically disengaged from the seal housing 180 as the needle 120 is removed from the vasculature and disengaged from the handle 170. Advantageously, the guidewire 130 is separated from the seal housing 180 while the user controls and manipulates the handle 170, allowing for greater control over the process.

Advantageously, the needle 120, tether 192, seal housing 180, needle hub 122 and blood flash system 140 assembly can be disengaged from the handle 170, guidewire 130, and catheter 150 assembly once vascular access is achieved, mitigating any obstruction to the advancement of the catheter 150 over the guidewire 130. Advantageously, the seal housing 180 encloses the needle tip 124 preventing any needle stick injuries once the needle 120 is disengaged from the handle 170.

In an embodiment, the threshold force required to disengage the seal housing 180 from the handle 170 can be significantly greater than the first force required to disengage the needle hub 122 from the handle 170 and withdraw the needle 120 proximally.

Advantageously, this ensures the seal housing 180 does not disengage the handle 170 until the needle tip 124 is disposed within the seal housing 180 and the tip shield 188 is deployed to the locked position, mitigating needle stick injuries. Further, the tether 192 allows the user to apply greater proximal forces to the needle hub 122 that is then transferred to the seal housing 180 by way of the tether 192.

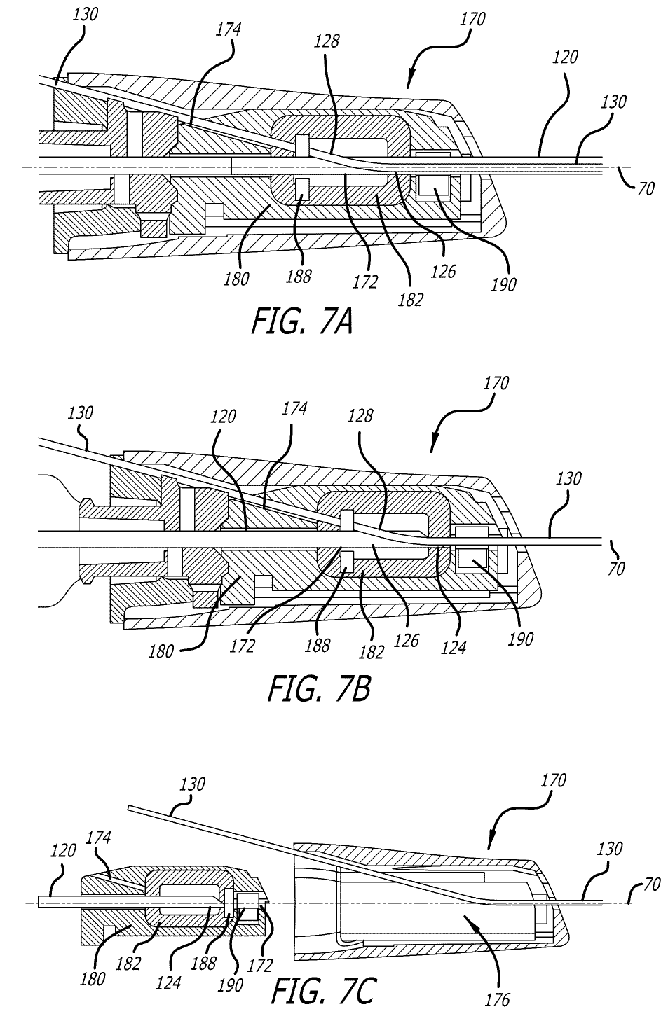

As shown in FIGS. 7A-7C, in an embodiment, the handle 170 includes a timing lock 190. To note, the timing lock 190 can be in place of, or in addition to, the detent 178. The timing lock 190 is configured to transition between a locked position and an unlocked position and is biased towards the unlocked position. In the locked position, the timing lock 190 is configured to secure the seal housing 180 within the recess 176 of the handle 170. In the unlocked position, the timing lock 190 allows the seal housing 180 to slide proximally relative to the handle 170.

In an embodiment, the timing lock 190 is disposed distally within the seal housing 180 and is aligned with a distal portion of the handle needle lumen 172. As shown in FIG. 7A, when a portion of the needle 120 is disposed within the distal portion of the handle needle lumen 172, the needle 120 maintains the timing lock 190 in the locked position.

As shown in FIGS. 7B-7C, as the needle 120 is withdrawn proximally, the needle tip 124 passes the timing lock 190 and into the seal housing 180. As such, when the needle tip 124 proximal of the timing lock 190, and is within the seal housing 180, the timing lock 190 transitions to the unlocked position, allowing the needle 120 and seal housing 180 assembly to disengage the handle 170, as described herein.

In an embodiment, the timing lock 190 is operably coupled to the tip shield 188. As such in a first position the tip shield 188 is unlocked allowing the needle 120 to slide relative to the seal housing 180, and the timing lock 190 is locked, preventing seal housing 180 from sliding relative to the handle 170. As the needle tip 124 is withdrawn into the seal housing 180, the tip shield 188/timing lock 190 assembly is released and transitions towards a second position. In an embodiment, the tip shield 188/timing lock 190 assembly is biased towards the second position. In the second position, the tip shield 188 is locked preventing any distal movement of the needle 120 relative to the seal housing 180, and the timing lock 190 is unlocked allowing the seal housing 180 to slide relative to the handle 170. The needle 120 and seal housing 180 can disengage the handle 170 and guidewire 130 assembly, as described herein. In an embodiment, the timing lock 190 and the tip shield 188 can be the same mechanism. In an embodiment, the timing lock 190 and the tip shield 188 can be separate mechanisms that operate co-currently. In an embodiment, the timing lock 190 and the tip shield 188 can be separate mechanisms that operate independently of each other and can operate based on the movement and/or position of the needle 120. Advantageously, the timing lock 190 in the first position prevents the seal housing 180 from disengaging the handle 170 irrespective of how much proximal force is applied to the needle hub 122. As such, the timing lock 190 can further mitigate premature disengagement of the seal housing 180 from the handle 170.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

What is claimed is:

1. A catheter placement system, comprising:
a catheter;
a needle supported by a needle hub and extending distally to a needle tip, the needle defining a needle lumen;
a guidewire;
a seal housing including a tip shield transitionable between a locked position and an unlocked position;
a tether extending between the needle hub and the seal housing;
a handle defining a recess and configured to slidably receive the seal housing therein, wherein the handle and the seal housing co-operate to define a handle needle lumen and a handle guidewire lumen; and
a detent configured to retain the seal housing within the recess when a first force is applied to the needle, and configured to deflect radially outwards to allow the seal housing to disengage the handle when a second force is applied.

2. The catheter placement system according to claim 1, wherein the first force is less than a threshold force required to deflect the detent, and the second force is equal to or greater than the threshold force.

3. The catheter placement system according to claim 1, wherein one or both of the first force and the second force urges the needle in a proximal direction.

4. The catheter placement system according to claim 1, wherein the tip shield in the locked position prevents distal movement of the tip of the needle relative to the seal housing, the tip shield being biased towards the locked position.

5. The catheter placement system according to claim 1, wherein the seal housing further includes a slot extending between an outer surface of the seal housing and one or both of the handle needle lumen and the handle guidewire lumen and configured to allow a portion of the guidewire to pass therethrough as the seal housing disengages the handle.

6. The catheter placement system according to claim 1, wherein the handle needle lumen extends between a distal end and a proximal end of the handle and the seal housing assembly and is configured to slidably receive a portion of the needle therein, and the handle guidewire lumen communicates with the handle needle lumen and extends at an angle therefrom.

7. The catheter placement system according to claim 6, wherein the seal housing includes a seal formed of a compliant material and defining a fluid tight seal extending about a junction between the handle needle lumen and the handle guidewire lumen.

8. The catheter placement system according to claim 7, wherein the needle includes a guidewire aperture communicating with the needle lumen and configured to align with the handle guidewire lumen when the needle hub engages a proximal end of the handle.

9. The catheter placement system according to claim 8, wherein the needle further includes a slot extending between the guidewire aperture and the needle tip.

10. The catheter placement system according to claim 1, wherein the catheter is a rapidly insertable central catheter and further includes a blood flash system coupled to the needle hub.

11. The catheter placement system according to claim 1, further including a support pin disposed within the handle and abutting against a portion of the guidewire to support the guidewire, the support pin causing the guidewire to automatically disengage the seal housing when the seal housing is urged proximally.

12. The catheter placement system according to claim 1, wherein the tether is configured to allow the needle hub to be withdrawn proximally up until a predetermined distance (d) where the tether prevents further proximal movement relative to the seal housing beyond the predetermined distance (d).

13. A method of placing a catheter, comprising:
forming an insertion site using a needle supported by a needle hub;
advancing a guidewire through a handle guidewire lumen of a handle, to a target location;
withdrawing the needle proximally through a handle needle lumen until a distal tip is located within a seal housing, the seal housing defining a junction between the handle needle lumen and the handle guidewire lumen;
transitioning a tip shield to a locked position to prevent further distal movement of the needle tip relative to the seal housing; and
urging the needle proximally to deflect a detent radially outwards and disengage the seal housing from the handle.

14. The method according to claim 13, wherein withdrawing the needle proximally further includes applying a first proximal force and urging the needle proximally to deflect the detent includes a second proximal force, the second proximal force being greater than the first proximal force and equal to or greater than a threshold force required to deflect the detent.

15. The method according to claim 14, wherein the seal housing is slidably engaged within a recess defined by the handle, the detent abutting against a portion of the seal housing to retain the seal housing within the recess when the first proximal force is applied.

16. The method according to claim 13, wherein the step of urging the needle proximally urges a portion of the guidewire, disposed within the handle guidewire lumen, through a slot extending between the handle guidewire lumen and an outer surface of the seal housing.

17. The method according to claim 13, further including a tether extending between the needle hub and the seal housing and configured to prevent further proximal movement of the needle hub relative to the seal housing beyond a predetermined distance (d).

18. A catheter placement system, comprising:
a needle defining a needle lumen extending between a needle hub and a needle tip;
a guidewire;
a seal housing;
a tether extending between the needle hub and the seal housing;
a handle defining a recess and configured to slidably receive the seal housing therein, wherein the handle and the seal housing co-operate to define a handle needle lumen and a handle guidewire lumen;
a tip shield transitionable between an unlocked position and a locked position; and
a timing lock disposed within the seal housing and transitionable between a first position and a second position, wherein, in the first position, the timing lock is configured prevent movement of the seal housing relative to the handle, and in the second position the timing lock is configured to allow the seal housing to slide relative to the handle.

19. The catheter placement system according to claim 18, wherein the timing lock is biased towards the second position and is retained in the first position by a portion of the needle disposed in a distal portion of the handle needle lumen.

20. The catheter placement system according to claim 18, wherein the timing lock is configured to transition from the first position to the second position when the needle tip is disposed within the seal housing.

21. The catheter placement system according to claim 18, wherein the timing lock in the second position allows the needle and the seal housing assembly to disengage from the guidewire and the handle assembly.

22. The catheter placement system according to claim 18, further including a support pin coupled with the handle and configured to support the guidewire as the seal housing slides proximally relative to the handle, the support pin causing the guidewire to automatically disengage the seal housing when the seal housing is urged proximally.
